(19) United States
(12) Patent Application Publication
(10) Pub. No.: US 2025/0092150 A1
(43) Pub. Date: Mar. 20, 2025

(54) FC POLYPEPTIDE VARIANTS HAVING AN INCREASED HALF-LIFE

(71) Applicant: LABORATOIRE FRANÇAIS DU FRACTIONNEMENT ET DES BIOTECHNOLOGIES, Puteaux (FR)

(72) Inventor: Céline MONNET, Lambersart (FR)

(21) Appl. No.: 18/953,705

(22) Filed: Nov. 20, 2024

Related U.S. Application Data
(62) Division of application No. 16/345,620, filed on Apr. 26, 2019.

(30) Foreign Application Priority Data
Oct. 28, 2016 (FR) 16 60495

(51) Int. Cl. C07K 16/28; C07K 16/34

(52) U.S. Cl. CPC C07K 16/2887; C07K 16/34; C07K 2317/41; C07K 2317/52; C07K 2317/90; C07K 2317/94; C07K 2319/30; C07K 2319/90

(57) ABSTRACT

Disclosed is a variant of a parent polypeptide including an Fc fragment, the variant having an improved half-life with respect to the parent polypeptide, and including at least one mutation of the Fc fragment increasing the binding of Fc to FcRn; and at least one mutation of the Fc fragment increasing the sialylation of Fc.

### CROSS REFERENCE TO RELATED APPLICATIONS

This application is a Divisional of application Ser. No. 16/345,620, filed on Apr. 26, 2019, which is the National Phase under 35 U.S.C. § 371 of International Application No. PCT/EP2017/077689, filed on Oct. 27, 2017, which claims the benefit under 35 U.S.C. § 119(a) to Patent Application No. 16 60495, filed in France on Oct. 28, 2016, all of which are hereby expressly incorporated by reference into the present application.

### REFERENCE TO AN ELECTRONIC SEQUENCE LISTING

The contents of the electronic sequence listing (7111-0500_SequenceListing.xml; Size: 15,760 bytes; and Date of Creation: Nov. 18, 2024) is herein incorporated by reference in its entirety.

### FIELD OF THE INVENTION

The present invention relates to a variant of a parent polypeptide comprising an Fc fragment with an improved half-life relative to the parent polypeptide. The invention also relates to a method for increasing the half-life of an Fc fragment.

### TECHNOLOGICAL BACKGROUND

Monoclonal antibodies are used today as therapeutic agents to treat a variety of conditions, including cancers, autoimmune diseases, chronic inflammatory diseases, transplant rejection, infectious diseases and cardio-vascular diseases. They therefore represent a major therapeutic issue. Many of them are already on the market, and an ever increasing proportion is under development or undergoing clinical trials. However, there is an important need to optimize the structural and functional properties of the antibodies, in order to control side effects.

One of the critical questions in the use of monoclonal antibodies in therapy is their persistence in the bloodstream. The clearance of the antibody directly affects the effectiveness of the treatment, and therefore the frequency and amount of drug delivery, which may cause adverse effects in the patient.

However, there is still a need to find antibodies, or fragments of antibodies, having an improved half-life thus making it possible to maintain their effectiveness and their biological properties of interest, with a lower dosage.

### DESCRIPTION OF THE INVENTION

The present invention provides means for obtaining a variant of a parent polypeptide comprising an Fc fragment having an improved half-life. The term “half-life” refers to the biological half-life of a polypeptide of interest in the circulation of a given patient, particularly human or murine, such as a mouse, and is represented by the time required for half of the amount of the polypeptide of interest present in the patient's circulation, to be eliminated from the circulation and/or other tissues of the patient. The half-life is calculated in particular as described in Example 2.

In fact, the inventors have surprisingly discovered that an Fc polypeptide, in particular an Fc fragment mutated at specific positions has a substantially increased half-life compared to the non-mutated Fc fragment. This thus makes it possible to increase the therapeutic properties of the Fc polypeptide or of the Fc fragment. In addition, the inventors have discovered that certain combinations of particular mutations have a synergistic effect on the increase of the half-life, further reinforcing the advantageous properties of Fc polypeptides, in particular of Fc fragments according to the invention.

Furthermore and surprisingly, the inventors have discovered that the half-life of a polypeptide comprising a mutated Fc fragment at specific positions that is increased relative to that of an unmutated parent polypeptide, is not correlated with an in vitro increase in the binding of the Fc fragment mutated to FcRn, compared to that of the non-mutated Fc fragment. Thus, one of the aims of the present invention is to be able to propose Fc polypeptides or Fc fragments having an increased half-life, independently of the binding to FcRn.

### FIGURES

FIG. 1 shows alignments of native human IgG1 sequences referring to positions 216 to 447 (according to the EU index) with the corresponding sequences of human IgG2 (SEQ ID NO: 7), human IgG3 (SEQ ID NO: 8) and human IgG4 (SEQ ID NO: 9). The IgG1 sequences refer to the G1m1, 17 allotype (SEQ ID NO: 6) and the G1m3 allotype (SEQ ID NO: 10). The “lower hinge CH2-CH3” domain of IgG1 begins with cysteine 226 (see arrow). The CH2 domain is highlighted in gray and the CH3 domain is italicized.

FIG. 2 shows the half-life of anti-CD20 antibodies (T5A_74 variants) produced in YB2/0 cells: The concentration of immunoglobulins in the serum of transgenic mice was evaluated over time.

FIG. 3 shows the half-life of anti-CD20 antibodies (C6A_74 variants) produced in YB2/0 cells: The concentration of immunoglobulins in the serum of transgenic mice was evaluated over time.

FIG. 4 shows the half-life of anti-CD20 antibodies (T5A_74 variants) produced in YB2/0 cells, before or after desialylation: The concentration of immunoglobulins in the serum of transgenic mice was evaluated over time.

FIG. 5 shows the hFcRn binding on cells of anti-CD20 antibodies (T5A_74 variants) produced in YB2/0 cells, before or after desialylation.

FIG. 6 shows the hFcRn binding on cells of anti-CD20 antibodies (C6A_74 variants) produced in YB2/0 cells, before or after desialylation.

FIG. 7 shows the CD20 binding on cells of anti-CD20 antibodies (T5A_74 variants) produced in YB2/0 cells, before or after desialylation.

FIG. 8 shows the CD20 binding on cells of anti-CD20 antibodies (C6A_74 variants) produced in YB2/0 cells, before or after desialylation.

### DEFINITIONS

As used herein, the terms “protein” and “polypeptide” are used interchangeably and refer to a sequence of at least two covalently linked amino acids, including proteins, polypeptides, oligopeptides, and peptides.

The terms “protein” and “polypeptide” comprise, in particular, antibodies or immunoglobulins, in particular whole, monoclonal, multi-specific, bi-specific, dual-specific, synthetic, chimeric, humanized, human, fusion proteins with immunoglobulins, conjugated antibodies, and their fragments.

The terms “protein” and “polypeptide” also include Fc polypeptides defined by a polypeptide comprising all or part of an Fc region, including isolated Fc, conjugated Fc, multimeric Fc fragments and Fc fusion proteins.

By “Fc fragment” or “Fc region” is meant the constant region of a full length immunoglobulin excluding the first immunoglobulin constant region domain (i.e. CH1-CL). Thus the Fc fragment refers to a homodimer, each monomer comprising the last two constant domains of IgA, IgD, IgG (i.e. CH2 and CH3), or the last three constant domains of IgE and IgM (i.e. CH2, CH3 and CH4), and the flexible N-terminal hinge region of these domains, in whole or in part. The Fc fragment, when it is derived from IgA or IgM, may comprise the J chain. Preferably, an Fc fragment of an IgG1, which consists of a part of the flexible N-terminal hinge and CH2-CH3 domains, is used in the present invention, i.e. the portion of amino acid C226 to the C-terminal, the numbering being indicated according to the EU index or equivalent in Kabat. Preferably, an Fc fragment of a human IgG1 (i.e. amino acids 226 to 447 is used according to the EU index or equivalent in Kabat). In this case, the part of the flexible N-terminal hinge is the lower hinge, which refers to positions 226 to 230, the CH2 domain refers to positions 231 to 340 while the CH3 domain refers to positions 341-447 according to the EU index or equivalent in Kabat. The fragment Fc used according to the invention may furthermore comprise all or part of the flexible N-terminal hinge (all corresponding to positions 216 to 230 according to the index EU), which includes the upper part of the N-terminal flexible hinge region upstream of the position 226. In this case, preferably, an Fc fragment of a human IgG1 comprising a portion of the region located between the positions 216 to 226 (according to the EU index) is used. In this case, the Fc fragment of the human IgG1 used corresponds to the residues of the position 216 to 447, 217 to 447, 218 to 447, 219 to 447, 220 to 447, 221 to 447, 222 to 447, 223 to 447, 224 to 447 or 225 to 447, wherein the numbering is according to the EU index or equivalent in Kabat. Preferably in this case, the native Fc fragment used corresponds to the residues of position 216 to 447, wherein the numbering is according to the EU index or equivalent in Kabat.

Preferably, the native Fc fragment used is chosen from the sequences SEQ ID NO: 1, 2, 3, 4 and 5. Preferably, the Fc fragment included in the parent polypeptide has the sequence SEQ ID NO: 1. The sequences represented in SEQ ID NO: 1, 2, 3, 4 and 5 are free of an N-terminal hinge region.

The sequences represented in SEQ ID NO: 6, 7, 8, 9 and 10 respectively correspond to the sequences represented in SEQ ID NO: 1, 2, 3, 4 and 5 with all of their N-terminal hinge regions. Also, in a particular embodiment, the Fc fragment included in the parent polypeptide is chosen from the sequences SEQ ID NO: 6, 7, 8, 9 and 10.

Preferably, the Fc fragment included in the parent polypeptide has a sequence consisting of amino acids at the positions 1-232, 2-232, 3-232, 4-232, 5-232, 6-232, 7-232, 8-232, 9-232, 10-232 or 11-232 of the sequence SEQ ID NO: 6.

The definition of “Fc fragment” includes an scFc fragment for “single chain Fc”. By “scFc fragment” is meant a single chain Fc fragment, obtained by genetic fusion of two Fc monomers linked by a polypeptide linker. The scFc folds naturally into a functional dimeric Fc region. Preferably, the Fc fragment used in the context of the invention is chosen from the Fc fragment of an IgG1 or IgG2. More preferably, the Fc fragment used is the Fc fragment of an IgG, preferentially an IgG1, more preferably of sequence SEQ ID NO: 1.

In the present application, the numbering of Fc residues is that of the EU index or equivalent in Kabat (Sequences of Proteins of Immunological Interest, 5th Ed. Public Health Service, National Institutes of Health, Bethesda, Md. (1991)).

By “amino acid mutation” is meant here a change in the amino acid sequence of a polypeptide. A mutation is chosen, in particular, from a substitution, an insertion and a deletion. By “substitution” is meant the replacement of one or more amino acids at a particular position in a parent polypeptide sequence by the same number of other amino acids. Preferably, the substitution is punctual, i.e. it concerns only one amino acid. For example, the N434S (also called 434S) substitution refers to a variant of a parent polypeptide, wherein the asparagine at position 434 of the Fc fragment according to the EU index or equivalent in Kabat is replaced by serine. By “insertion” is meant the addition of at least one amino acid at a particular position in a parent polypeptide sequence. For example, insertion G>235-236 refers to a glycine insertion between positions 235 and 236. By “deletion” is meant the removal of at least one amino acid at a particular position in a parent polypeptide sequence. For example, E294del refers to the removal of glutamic acid at position 294; such a deletion may also be called Del294 or del294.

By “parent polypeptide” is meant a reference polypeptide which is subsequently modified to generate a variant. The parent polypeptide may be a naturally occurring polypeptide, a variant of a naturally occurring polypeptide, a modified version of a natural polypeptide, or a synthetic polypeptide.

By “variant” is meant a polypeptide sequence which is different from the parent polypeptide sequence by at least one amino acid modification. Preferably, the sequence of the variant is at least 80% identical with the sequence of the parent polypeptide, and more preferably at least 90%, 91%, 92%, 93%, 94%, 95%, 96%, 97%, 98%, 99% or 99.5% identical. By “percentage of identity” between two amino acid sequences in the sense of the present invention, is meant the designation of a percentage of identical amino acid residues between the two sequences to be compared, obtained after the best alignment and over the entire length of the variant sequence, this percentage being purely statistical and the differences between the two sequences being randomly distributed over their entire length. The identity calculation takes place here over the entire length of the variant sequence, and excludes a calculation on a partial length. By “best alignment” or “optimal alignment” is meant the alignment for which the percentage of identity determined as hereinafter is the highest. Sequence comparisons between two amino acid sequences are traditionally performed by comparing these sequences after optimally aligning them, the comparison being made by segment or by “comparison window” to identify and compare the local regions of sequence similarity. The optimal alignment of the sequences for comparison may be realized, besides manually, by means of the local homology algorithm of Smith and Waterman (1981, J. Mol Evol., 18:38-46), by means of the local homology algorithm of Neddleman and Wunsch (1970), using the similarity search method of Pearson and Lipman (1988, PNAS, 85:2444-2448), using computer programs using these algorithms (GAP, BESTFIT, BLAST P, BLAST N, FASTA and TFASTA in the Wisconsin Genetics Software Package, Genetics Computer Group, 575 Science Dr., Madison, WI).

By “sialylation” is meant the glycosylation mechanism corresponding to an addition, by covalent bonding, of at least one sialic acid (i.e. N-acetylneuraminic acid and its derivatives, such as N-glycosylneuraminic acid, N-acid). acetylglycoylneuraminic) in the glycosylated chain of the protein.

By “Fc region receptor” or “FcR” is meant, in particular, C1q and Fcγ receptors (FcγR). The “Fcγ” or “FcγR” receptors refer to the IgG-type immunoglobulin receptors, called CD64 (FcγRI), CD32 (FcγRII), and CD16 (FcγRIII), in particular to the five FcyRIa, FcyRIIa, FcyRIIb, FcγRIIIa and FcγRIIb expressed receptors,. All are activating receptors for effector cells carrying such Fc receptors, except for human FcγRIIb which is an inhibitory receptor for the activation of immune cells (Muta T et al., Nature, 1994, 368:70-73).

By “FcRn” or “neonatal Fc receptor” as used herein is meant a protein that binds to the Fc region of IgG and is at least partially encoded by an FcRn gene. FcRn may be from any organism including, but not limited to, humans, mice, rats, rabbits and monkeys. As is known in the art, the functional FcRn protein comprises two polypeptides, often referred to as heavy chain and light chain. The light chain is beta-2-microglobulin while the heavy chain is encoded by the FcRn gene. Unless otherwise noted here, FcRn or FcRn protein refers to the a chain complex with beta-2-microglobulin. In humans, the gene encoding FcRn is called FCGRT.

### Fc Fragment Variants

The object of the present invention is a variant of a parent polypeptide comprising an Fc fragment, the variant having an improved half-life with respect to the parent polypeptide, and comprising:
At least one mutation of the Fc fragment increasing the sialylation of Fc; and
At least one mutation of the Fc fragment increasing the binding of Fc to FcRn.

The present invention also relates to a variant of a parent polypeptide comprising an Fc fragment, the variant having an improved half-life with respect to the parent polypeptide, and comprising at least three mutations with respect to the Fc fragment of the parent polypeptide, comprising:
a mutation A) of at least one amino acid chosen from amino acids in position 240, 241, 242, 243, 258, 259, 260, 261, 262, 263, 264, 265, 266, 267, 290, 291, 292, 293, 294, 295, 296, 298, 299, 300, 301, 302, 303, 304 or 305 of the Fc fragment; and
a mutation B) selected from the group consisting of 378V, 378T, 434Y and 434S; and
at least one C) mutation selected from the group consisting of 226G, 228L, 228R, 230S, 230T, 230L, 241L, 264E, 307P, 315D, 330V, 362R, 378V, 378T, 389T, 389K, 434Y and 434S,
it being understood that:
mutations A, B and C do not take place on the same amino acid, and
the amino acid position number of the Fc fragment is that of the EU index or equivalent in Kabat.

By “mutations A, B and C do not occur on the same amino acid” is meant that each of mutations A, B and C is performed on a different amino acid. In other words, at least 3 distinct amino acids are mutated in the variants according to the invention.

Preferably, the mutation A) is del294 or 264E.

In fact, the mutants according to the invention, in particular comprising the combinations of mutations 294del/N315D/A330V/N361D/A378V/N434Y, V264E/N315D/A330V/N361D/A378V/N434Y or 294del/259I/315D/434Y, exhibit an increased half-life, regardless of the FcRn binding.

In addition, as shown in the examples, the single mutation Del294 increases the half-life, but not the MRT (i.e. mean residence time in the body), compared to WT. On the other hand, the half-life and the MRT are both strongly increased with a variant comprising both the Del294 mutation (mutation A), but also a combination of mutations B) and C).

In a particular embodiment, the variant comprises at least one combination of mutations selected from the group consisting of 226G/315D/434Y, 230S/315D/434Y, 230T/315D/434Y, 230T/264E/434S, 230T/389T/434S, 241L/264E/378V, 241L/264E/434S, 250A/389K/434Y, 259I/315D/434Y, 264E/378T/396L, 264E/378V/416K, 264E/378V/434S, 264E/396L/434S, 294del/307P/434Y, 307P/378V/434Y, 315D/330V/434Y, 315D/382V/434Y and 378V/383N/434Y, it being understood that the amino acid position numbering of the Fc fragment is that of the EU index or equivalent in Kabat.

Thus, preferably, the variant comprises:
i) a mutation A of at least one amino acid selected from amino acids in positions 240, 241, 242, 243, 258, 259, 260, 261, 262, 263, 264, 265, 266, 267, 290, 291 292, 293, 294, 295, 296, 298, 299, 300, 301, 302, 303, 304 or 305; and
ii) at least one combination of mutations selected from the group consisting of 226G/315D/434Y, 230S/315D/434Y, 230T/315D/434Y, 230T/264E/434S, 230T/389T/434S, 241L/264E/378V, 241L/264E/434S, 250A/389K/434Y, 259I/315D/434Y, 264E/378T/396L, 264E/378V/416K, 264E/378V/434S, 264E/396L/434S, 294del/307P/434Y, 307P/378V/434Y, 315D/330V/434Y, 315D/382V/434Y et 378V/383N/434Y, it being understood that mutation A can not take place on the same amino acid as one of the amino acids of mutation ii), and it being understood that the amino acid position numbering of the Fc fragment is that of the EU index or equivalent in Kabat.

In a particular embodiment, the variant further comprises at least one mutation selected from the group consisting of 226G, 227L, 230S, 230T, 230L, 231T, 241L, 243L, 250A, 256N, 259I, 264E, 265G, 267R, 290E, 294del, 303A, 305A, 307P, 307A, 3081, 315D, 322R, 325S, 327V, 330V, 342R, 347R, 352S, 361D, 362R, 362E, 370R, 378V, 378T, 382V, 383N, 386R, 386K, 387T, 389T, 389K, 392R, 395A, 396L, 397M, 403T, 404L, 415N, 416K, 421T, 426T, 428L, 433R, 434Y, 434S and 439R, it being understood that the amino acid position numbering of the Fc fragment is that of the EU index or equivalent in Kabat.

Thus, preferably, the variant comprises:
i) a mutation A of at least one amino acid selected from amino acids in positions 240, 241, 242, 243, 258, 259, 260, 261, 262, 263, 264, 265, 266, 267, 290, 291 292, 293, 294, 295, 296, 298, 299, 300, 301, 302, 303, 304 or 305; and
ii) at least one combination of mutations selected from the group consisting of 226G/315D/434Y, 230S/315D/434Y, 230T/315D/434Y, 230T/264E/434S, 230T/389T/434S, 241L/264E/378V, 241L/264E/434S, 250A/389K/434Y, 259I/315D/434Y, 264E/378T/396L, 264E/378V/416K, 264E/378V/434S, 264E/396L/434S, 294del/307P/434Y, 307P/378V/434Y, 315D/330V/434Y, 315D/382V/434Y and 378V/383N/434Y, and at least one mutation selected from the group consisting of 226G, 227L, 230S, 230T, 230L, 231T, 241L, 243L, 250A, 256N, 259I, 264E, 265G, 267R, 290E, 294del, 303A, 305A, 307P, 307A., 3081, 315D, 322R, 325S, 327V, 330V, 342R, 347R, 352S, 361D, 362R, 362E, 370R, 378V, 378T, 382V, 383N, 386R, 386K, 387T, 389T, 389K, 392R, 395A, 396L, 397M, 403T, 404L, 415N, 416K, 421T, 426T, 428L, 433R, 434Y, 434S and 439R, it being understood that mutation A can not take place on the same amino acid as one of the amino acids of mutation ii), and it being understood that the amino acid position numbering of the Fc fragment is that of the EU index or equivalent in Kabat.

In a particular embodiment, the variant comprises at least one combination of mutations ii) selected from the group consisting of 307A/315D/330V/382V/389T/434Y, 256N/378V/383N/434Y, 315D/330V/361D/378V/434Y, 259I/315D/434Y, 230S/315D/428L/434Y, 241L/264E/307P/378V/433R, 250A/389K/434Y, 305A/315D/330V/395A/434Y, 264E/386R/396L/434S/439R, 315D/330V/362R/434Y, 294del/307P/434Y, 305A/315D/330V/389K/434Y, 315D/327V/330V/397M/434Y, 230T/241L/264E/265G/378V/421T, 264E/396L/415N/434S, 227L/264E/378V/434S, 264E/378T/396L, 230T/315D/362R/426T/434Y, 226G/315D/330V/434Y, 230L/241L/243L/264E/307P/378V, 250A/315D/325S/330V/434Y, 290E/315D/342R/382V/434Y, 241L/315D/330V/392R/434Y, 241L/264E/307P/378V/434S, 230T/264E/403T/434S, 264E/378V/416K, 230T/315D/362E/434Y, 226G/315D/434Y, 226G/315D/362R/434Y, 226G/264E/347R/370R/378V/434S, 3081/315D/330V/382V/434Y, 230T/264E/378V/434S, 231T/241L/264E/378T/397M/434S, 230L/264E/378V/434S, 230T/315D/330V/386K/434Y, 226G/315D/330V/389T/434Y, 267R/307P/378V/421T/434Y, 230S/315D/387T/434Y, 230S/264E/352S/378V/434S et 230T/303A/322R/389T/404L/434S, it being understood that the amino acid position numbering of the Fc fragment is that of the EU index or equivalent in Kabat.

Thus, preferably, a variant according to the invention comprises:
i) a mutation A of at least one amino acid selected from amino acids in positions 240, 241, 242, 243, 258, 259, 260, 261, 262, 263, 264, 265, 266, 267, 290, 291 292, 293, 294, 295, 296, 298, 299, 300, 301, 302, 303, 304 or 305; and
ii) at least one combination of mutations selected from the group consisting of 307A/315D/330V/382V/389T/434Y, 256N/378V/383N/434Y, 315D/330V/361D/378V/434Y, 259I/315D/434Y, 230S/315D/428L/434Y, 241L/264E/307P/378V/433R, 250A/389K/434Y, 305A/315D/330V/395A/434Y, 264E/386R/396L/434S/439R, 315D/330V/362R/434Y, 294del/307P/434Y, 305A/315D/330V/389K/434Y, 315D/327V/330V/397M/434Y,230T/241L/264E/265G/378V/421T, 264E/396L/415N/434S, 227L/264E/378V/434S, 264E/378T/396L, 230T/315D/362R/426T/434Y, 226G/315D/330V/434Y,230L/241L/243L/264E/307P/378V, 250A/315D/325S/330V/434Y, 290E/315D/342R/382V/434Y, 241L/315D/330V/392R/434Y, 241L/264E/307P/378V/434S, 230T/264E/378V/434S, 264E/378V/416K, 230T/315D/362E/434Y, 226G/315D/434Y, 226G/315D/330V/389T/434Y, 226G/315D/362R/434Y, 308I/315D/330V/382V/434Y, 230T/264E/403T/434S, 231T/241L/264E/378T/397M/434S, 230L/264E/378V/434S, 230T/315D/330V/386K/434Y, 226G/264E/347R/370R/378V/434S, 267R/307P/378V/421T/434Y, 230S/315D/387T/434Y, 230S/264E/352S/378V/434S and 230T/303A/322R/389T/404L/434S,
it being understood that mutation A can not take place on the same amino acid as one of the amino acids of mutation ii), and it being understood that the amino acid position numbering of the Fc fragment is that of the EU index or equivalent in Kabat.

In a particular embodiment, the variant comprises at least one combination of mutations ii) selected from 307A/315D/330V/382V/389T/434Y, 256N/378V/383N/434Y, 259I/315D/434Y, 230S/315D/428L/434Y, 294del/307P/434Y et 315D/330V/361D/378V/434Y, it being understood that the amino acid position numbering of the Fc fragment is that of the EU index or equivalent in Kabat.

In a particular embodiment, the variant comprises at least one combination of mutations ii) selected from 256N/378V/383N/434Y, 259I/315D/434Y and 315D/330V/361D/378V/434Y, it being understood that the numbering of amino acid positions of the Fc fragment is that of the EU index or equivalent in Kabat.

In a particular embodiment, the variant comprises a mutation A) selected from del294 and 264E, and at least one combination of mutations ii) selected from 256N/378V/383N/434Y, 259I/315D/434Y and 315D/330V/361D/378V/434Y, it being understood that the amino acid position numbering of the Fc fragment is that of the EU index or equivalent in Kabat.

Preferably, the half-life of the variant is increased by a factor of at least 2, preferably greater than 3, preferably greater than 4, preferably greater than 5, preferably greater than 6, preferably greater than 8 preferably greater than 9, preferably greater than 10, preferably greater than 15, preferably greater than 20, preferably greater than 25, and preferably greater than 30 relative to the parent polypeptide.

Preferably, the variant according to the invention is obtained by production in cells, in particular YB2/0 cells, or by production in transgenic mammals, as described below in the section “Method of increasing the half-life of an Fc fragment.

Preferably, the parent polypeptide comprises an Fc fragment selected from wild-type Fc fragments, their fragments and their natural variants. Preferably, according to an First alternative, the parent polypeptide consists of an Fc fragment, and preferably an entire Fc fragment.

Preferably, according to a second alternative, the parent polypeptide consists of an amino acid sequence fused in N- or C-terminal to an Fc fragment. In this case, advantageously, the parent polypeptide is an immunoglobulin or an antibody, an Fc fusion polypeptide, or an Fc conjugate.

Preferably, the Fc fragment of the parent polypeptide is chosen from the sequences SEQ ID NO: 1, 2, 3, 4 and 5. Preferably, the Fc fragment of the parent polypeptide consists of the sequence SEQ ID NO: 1. The sequences represented in SEQ ID NO: 1, 2, 3, 4 and 5 are free of the upper part of the N-terminal hinge region.

The sequences represented in SEQ ID NO: 6, 7, 8, 9 and 10 respectively correspond to the sequences represented in SEQ ID NOs: 1, 2, 3, 4 and 5 with their N-terminal hinge regions. Also, in a particular embodiment, the Fc fragment of the parent polypeptide is chosen from the sequences SEQ ID NO: 6, 7, 8, 9 and 10.

| SEQ | | |
|---|---|---|
| ID | | |
| NO: | Protein | Sequence |
| 1 | Fc region of human IgG1 | CPPCPAPELLGGPSVFLFPPKPKDTLMISRTPEVTCVV |
| | G1m1,17 (residues 226- | VDVSHEDPEVKFNWYVDGVEVHNAKTKPREEQYNST |
| | 447 according to the EU | YRVVSVLTVLHQDWLNGKEYKCKVSNKALPAPIEKTIS |
| | index or equivalent in | KAKGQPREPQVYTLPPSRDELTKNQVSLTCLVKGFYP |
| | Kabat) without upper N- | SDIAVEWESNGQPENNYKTTPPVLDSDGSFFLYSKLT |
| | terminal hinge region | VDKSRWQQGNVFSCSVMHEALHNHYTQKSLSLSPGK |
| 2 | Fc region of human IgG2 | CPPCPAPPVAGPSVFLFPPKPKDTLMISRTPEVTCVVV |
| | without upper N-terminal | DVSHEDPEVQFNWYVDGVEVHNAKTKPREEQFNSTF |
| | hinge region | RVVSVLTVVHQDWLNGKEYKCKVSNKGLPAPIEKTISK |
| | | TKGQPREPQVYTLPPSREEMTKNQVSLTCLVKGFYPS |
| | | DIAVEWESNGQPENNYKTTPPMLDSDGSFFLYSKLTV |
| | | DKSRWQQGNVFSCSVMHEALHNHYTQKSLSLSPGK |
| 3 | Fc region of human IgG3 | CPRCPAPELLGGPSVFLFPPKPKDTLMISRTPEVTCVV |
| | without upper N-terminal | VDVSHEDPEVQFKWYVDGVEVHNAKTKPREEQYNST |
| | hinge region | FRVVSVLTVLHQDWLNGKEYKCKVSNKALPAPIEKTIS |
| | | KTKGQPREPQVYTLPPSREEMTKNQVSLTCLVKGFYP |
| | | SDIAVEWESSGQPENNYNTTPPMLDSDGSFFLYSKLT |
| | | VDKSRWQQGNIFSCSVMHEALHNRFTQKSLSLSPGK |
| 4 | Fc region of human IgG4 | CPSCPAPEFLGGPSVFLFPPKPKDTLMISRTPEVTCVV |
| | without upper N-terminal | VDVSQEDPEVQFNWYVDGVEVHNAKTKPREEQFNST |
| | hinge region | YRVVSVLTVLHQDWLNGKEYKCKVSNKGLPSSIEKTIS |
| | | KAKGQPREPQVYTLPPSQEEMTKNQVSL |
| | | TCLVKGFYPSDIAVEWESNGQPENNYKTTPPVLDSDG |
| | | SFFLYSRLTVDKSRWQEGNVFSCSVMHEALHNHYTQ |
| | | KSLSLSLGK |
| 5 | Fc region human IgG1 | CPPCPAPELLGGPSVFLFPPKPKDTLMISRTPEVTCVV |
| | G1m3 without upper N- | VDVSHEDPEVKFNWYVDGVEVHNAKTKPREEQYNST |
| | terminal hinge region | YRVVSVLTVLHQDWLNGKEYKCKVSNKALPAPIEKTIS |
| | | KAKGQPREPQVYTLPPSREEMTKNQVSLTCLVKGFYP |
| | | SDIAVEWESNGQPENNYKTTPPVLDSDGSFFLYSKLT |
| | | VDKSRWQQGNVFSCSVMHEALHNHYTQKSLSLSPGK |
| 6 | Fc region of human IgG1 | EPKSCDKTHTCPPCPAPELLGGPSVFLFPPKPKDTLMI |
| | G1m1,17 with N-terminal | SRTPEVTCVVVDVSHEDPEVKFNWYVDGVEVHNAKT |
| | upper hinge region | KPREEQYNSTYRVVSVLTVLHQDWLNGKEYKCKVSN |
| | (residues 216-447 | KALPAPIEKTISKAKGQPREPQVYTLPPSR |
| | according to the EU | DELTKNQVSLTCLVKGFYPSDIAVEWESNGQPENNYK |
| | index or equivalent in | TTPPVLDSDGSFFLYSKLTVDKSRWQQGNVFSCSVM |
| | Kabat) | HEALHNHYTQKSLSLSPGK |
| 7 | Fc region of human IgG2 | ERKCCVECPPCPAPPVAGPSVFLFPPKPKDTLMISRTP |
| | with upper N-terminal | EVTCVVVDVSHEDPEVQFNWYVDGVEVHNAKTKPRE |
| | hinge region | EQFNSTFRVVSVLTVVHQDWLNGKEYKCKVSNKGLPA |
| | | PIEKTISKTKGQPREPQVYTLPPSREEMTKNQVSLTCL |
| | | VKGFYPSDIAVEWESNGQPENNYKTTPPMLDSDGSFF |
| | | LYSKLTVDKSRWQQGNVFSCSVMHEALHNHYTQKSL |
| | | SLSPGK |
| 8 | Fc region of human IgG3 | ELKTPLGDTTHTCPRCPEPKSCDTPPPCPRCPEPKSC |
| | with upper N-terminal | DTPPPCPRCPEPKSCDTPPPCPRCPAPELLGGPSVFL |
| | hinge region | FPPKPKDTLMISRTPEVTCVVVDVSHEDPEVQFKWYV |
| | | DGVEVHNAKTKPREEQYNSTFRVVSVLTV |
| | | LHQDWLNGKEYKCKVSNKALPAPIEKTISKTKGQPREP |
| | | QVYTLPPSREEMTKNQVSLTCLVKGFYPSDIAVEWES |
| | | SGQPENNYNTIPPMLDSDGSFFLYSKLTVDKSRWQQ |
| | | GNIFSCSVMHEALHNRFTQKSLSLSPGK |
| 9 | Fc region of human IgG4 | ESKYGPPCPSCPAPEFLGGPSVFLFPPKPKDTLMISRT |
| | with upper N-terminal | PEVTCVVVDVSQEDPEVQFNWYVDGVEVHNAKTKPR |
| | hinge region | EEQFNSTYRVVSVLTVLHQDWLNGKEYKCKVSNKGLP |
| | | SSIEKTISKAKGQPREPQVYTLPPSQEEM |
| | | TKNQVSLTCLVKGFYPSDIAVEWESNGQPENNYKTTP |
| | | PVLDSDGSFFLYSRLTVDKSRWQEGNVFSCSVMHEAL |
| | | HNHYTQKSLSLSLGK |
| 10 | Fc region of human IgG1 | EPKSCDKTHTCPPCPAPELLGGPSVFLFPPKPKDTLMI |
| | with upper N-terminal | SRTPEVTCVVVDVSHEDPEVKFNWYVDGVEVHNAKT |
| | hinge region | KPREEQYNSTYRVVSVLTVLHQDWLNGKEYKCKVSN |
| | | KALPAPIEKTISKAKGQPREPQVYTLPPSR |
| | | EEMTKNQVSLTCLVKGFYPSDIAVEWESNGQPENNYK |
| | | TTPPVLDSDGSFFLYSKLTVDKSRWQQGNVFSCSVM |
| | | HEALHNHYTQKSLSLSPGK |

Alternatively, the parent polypeptide may consist of an immunoglobulin, an antibody or an amino acid sequence fused at N- or C-terminal to an antibody or immunoglobulin. Preferably, the parent polypeptide is an immunoglobulin or an antibody.

### Method for Increasing the Half-Life of an Fc Fragment

The object of the present invention is also a method for increasing the half-life of a polypeptide comprising an Fc fragment, comprising the following steps:
Inserting at least one mutation of the Fc fragment increasing the sialylation of Fc; and
Inserting at least one mutation of the Fc fragment increasing the binding of Fc to FcRn.

The object of the present invention is also a method for increasing the half-life of a polypeptide comprising an Fc fragment, comprising the following steps:
i) insertion of a mutation A of at least one amino acid selected from amino acids in position 240, 241, 242, 243, 258, 259, 260, 261, 262, 263, 264, 265, 266, 267, 290, 291, 292, 293, 294, 295, 296, 298, 299, 300, 301, 302, 303, 304 or 305;
ii) insertion of a mutation B selected from the group consisting of 378V, 378T, 434Y and 434S and
insertion of at least one mutation C selected from the group consisting of 226G, 228L, 228R, 230S, 230T, 230L, 241L, 264E, 307P, 315D, 330V, 362R, 378V, 378T, 389T, 389K, 434Y and 434S;
it being understood that:
the mutations are carried out on the Fc fragment of the polypeptide, the mutations A, B and C do not take place on the same amino acid, and the amino acid position numbering of the Fc fragment is that of the EU index or equivalent in Kabat.

Preferably, the step i) is a deletion step of the amino acid at position 294 or an insertion step of a mutation 264E, it being understood that the numbering of the amino acid positions of the Fc fragment is that of the EU index or equivalent in Kabat.

Preferably, the step ii) consists of the insertion of a combination of mutations selected from the group consisting of 226G/315D/434Y, 230S/315D/434Y, 230T/315D/434Y, 230T/264E/434S, 230T/389T/434S, 241L/264E/378V, 241L/264E/434S, 250A/389K/434Y, 259I/315D/434Y, 378T/396L, 378V/416K, 378V/434S, 396L/434S, 307P/434Y, 307P/378V/434Y, 315D/330V/434Y, 315D/382V/434Y and 378V/383N/434Y,

it being understood that the amino acid position numbering of the Fc fragment is that of the EU index or equivalent in Kabat. Preferably, the step ii) consists of the insertion of a combination of mutations selected from the group consisting of 226G/315D/434Y, 230S/315D/434Y, 230T/315D/434Y, 230T/264E/434S, 230T/389T/434S, 241L/264E/378V, 241L/264E/434S, 250A/389K/434Y, 259I/315D/434Y, 264E/378T/396L, 264E/378V/416K, 264E/378V/434S, 264E/396L/434S, 294del/307P/434Y, 307P/378V/434Y, 315D/330V/434Y, 315D/382V/434Y and 378V/383N/434Y.

In a particular embodiment, the method further comprises a step iii) of inserting at least one mutation selected from the group consisting of 226G, 227L, 230S, 230T, 230L, 231T, 241L, 243L, 250A, 256N, 259I, 264E, 265G, 267R, 290E, 294del, 303A, 305A, 307P, 307A, 308I, 315D, 322R, 325S, 327V, 330V, 342R, 347R, 352S, 361D, 362R, 362E, 370R, 378V, 378T, 382V, 383N, 386R, 386K, 387T, 389T, 389K, 392R, 395A, 396L, 397M, 403T, 404L, 415N, 416K, 421T, 426T, 428L, 433R, 434Y, 434S and 439R, it being understood that the amino acid position numbering of the Fc fragment is that of the EU index or equivalent in Kabat.

Preferably, the step ii) consists in the insertion of a combination of mutations chosen from 307A/315D/330V/382V/389T/434Y, 256N/378V/383N/434Y, 259I/315D/434Y, 230S/315D/428L/434Y, 294del/307P/434Y and 315D/330V/361D/378V/434Y, it being understood that the amino acid position numbering of the Fc fragment is that of the EU index or equivalent in Kabat.

Preferably, the step ii) consists in inserting a combination of mutations selected from 256N/378V/383N/434Y, 259I/315D/434Y and 315D/330V/361D/378V/434Y, it being understood that the number of amino acid positions of the Fc fragment is that of the EU index or equivalent in Kabat.

Preferably, the method of increasing the half-life according to the invention is such that step i) is a deletion step of the amino acid in position 294, while step ii) consists in the insertion a combination of 315D/330V/361D/378V/434Y mutations.

All previously mentioned technical features are applicable here.

In preferred embodiments, the parent polypeptide is an immunoglobulin or an antibody, preferably an IgG, and the variant according to the invention is then selected from IgG variants. More preferably, the variant according to the invention is chosen from human IgG1, IgG2, IgG3 and IgG4 variants.

Preferably, the method for increasing the half-life of an Fc fragment according to the invention makes it possible to increase the half-life by a factor of at least 2, preferably greater than 3, preferably greater than 4, preferably greater than 5, preferably greater than 6, preferably greater than 8, preferably greater than 9, preferably greater than 10, preferably greater than 15, preferably greater than 20, preferably greater than 25, and preferably greater than 30, with respect to the half-life of the polypeptide comprising an Fc fragment before the mutation steps of the method.

More preferably, the method mutation step is obtained as follows:
a) providing a nucleic sequence encoding the parent polypeptide comprising the Fc fragment;
b) modifying the nucleic sequence provided in a) to obtain a nucleic sequence encoding the variant; and
c) expressing the nucleic sequence obtained in b) in a host cell, and recovering the variant.

Such a mutation step is therefore performed using a nucleic sequence (polynucleotide or nucleotide sequence) encoding the parent polypeptide (step a)). The nucleic acid sequence encoding the parent polypeptide may be synthesized chemically (Young L. and Dong, 2004, -Nucleic Acids Res., April 15; 32 (7), Hoover, DM and Lubkowski, J. 2002, Nucleic Acids Res., 30, Villalobos A, et al., 2006. BMC Bioinformatics, June 6; 7: 285). The nucleotide sequence encoding the parent polypeptide may also be amplified by PCR using suitable primers. The nucleotide sequence encoding the parent polypeptide may also be cloned into an expression vector. The DNA coding for such a parent polypeptide is inserted into an expression plasmid and inserted into an ad hoc cell line for its production (for example the HEK-293 FreeStyle line, the YB2/O line, or the CHO line). the protein thus produced is then purified by chromatography.

These techniques are described in detail in the reference manuals: Molecular cloning: a laboratory manual, 3rd edition-Sambrook and Russel eds. (2001) and Current Protocols in Molecular Biology—Ausubel et al. eds (2007).

The nucleic sequence provided in a) (polynucleotide), which encodes the parent polypeptide, is then modified to obtain a nucleic sequence encoding the variant. This is step b).

This step is the actual mutation stage. It may be performed by any known method of the prior art, in particular by directed mutagenesis or by random mutagenesis. Preferably, the random mutagenesis as described in the application WO02/038756 is used: it is the Mutagen technique. This technique uses a human DNA mutase, in particular chosen from DNA polymerases β, η and ι. A step of selecting mutants having retained FcRn binding is necessary to retain the mutants of interest.

Alternatively, amino acid substitutions are preferably made by site-directed mutagenesis, using the assembly PCR technique using degenerate oligonucleotides (see, for example, Zoller and Smith, 1982, Nucl Acids Res., 10:6487-6500. Kunkel, 1985, Proc Natl Acad Sci USA 82:488).

Finally, in step c), the nucleic sequence obtained in b) is expressed in a host cell, and the variant thus obtained is recovered.

The cellular host may be chosen from prokaryotic or eukaryotic systems, for example bacterial cells, but also yeast cells or animal cells, in particular mammalian cells. Insect cells or plant cells may also be used.

The preferred host cells are the YB2/0 rat line, the CHO hamster line, in particular the CHO dhfr- and CHO Lec13, PER.C6™ (Crucell) lines, the HEK cells in particular HEK293 (ATCC #CRL1573), T1080, EB66, K562, NSO, SP2/0, HeLa, BHK or COS cells. More preferably, the YB2/0 rat line is used.

The present invention therefore also relates to a method for producing a variant of a parent polypeptide comprising an Fc fragment, the variant having an improved half-life compared to the parent polypeptide, comprising the following steps:
a) providing a nucleic sequence encoding the parent polypeptide comprising the Fc fragment;
b) modifying the nucleic sequence provided in a) to obtain a nucleic sequence encoding the variant; and
c) expressing the nucleic sequence obtained in b) in a host cell YB2/0, and recovering the variant,
wherein step b) comprises:
i) insertion of a mutation A of at least one amino acid selected from amino acids in position 240, 241, 242, 243, 258, 259, 260, 261, 262, 263, 264, 265, 266, 267, 290, 291, 292, 293, 294, 295, 296, 298, 299, 300, 301, 302, 303, 304 or 305;

ii) insertion of a mutation B selected from the group consisting of 378V, 378T, 434Y and 434S and
insertion of at least one mutation C selected from the group consisting of 226G, 228L, 228R, 230S, 230T, 230L, 241L, 264E, 307P, 315D, 330V, 362R, 378T, 389T, 389K and 434S;
it being understood that:
the mutations are carried out on the Fc fragment of the polypeptide,
the mutations A, B and C do not take place on the same amino acid, and
the amino acid position numbering of the Fc fragment is that of the EU index or equivalent in Kabat.

More preferably, such a production method comprises, in step b):
(i) insertion of a mutation A selected from del294 and 264E;
(ii) insertion of a mutation B selected from 378V and 434Y, and insertion of at least one C mutation selected from 315D and 330V, preferably the insertion of at least C, 315D and 330V mutations,
it being understood that:
the mutations are carried out on the Fc fragment of the polypeptide,
the mutations A, B and C do not take place on the same amino acid, and the amino acid position numbering of the Fc fragment is that of the EU index or equivalent in Kabat.

More preferably, such a production method comprises, in step b), the insertion of a combination of mutations chosen from 259I/315D/434Y and 315D/330V/361D/378V/434Y, it being understood that the number of amino acid positions of the Fc fragment is that of the EU index or equivalent in Kabat.

Alternatively, the host cells may be transgenic animal cells modified to produce the polypeptide in the milk. In this case, the expression of a DNA sequence coding for the polypeptide according to the invention is controlled by a mammalian casein promoter or a mammalian whey promoter, the promoter not naturally controlling the transcription of the gene, and the DNA sequence further containing a secretion sequence of the protein. The secretion sequence comprises a secretion signal interposed between the coding sequence and the promoter. The animal may thus be chosen from sheep, goat, rabbit, sheep or cow.

The present invention therefore also relates to a method for producing a variant of a parent polypeptide comprising an Fc fragment, the variant having an improved half-life compared to the parent polypeptide, comprising the following steps:
a) providing a nucleic sequence encoding the parent polypeptide comprising the Fc fragment;
b) modifying the nucleic sequence provided in a) to obtain a nucleic sequence encoding the variant; and
c) expressing the nucleic sequence obtained in b) in a host cell selected from modified transgenic animal cells to produce the polypeptide in the milk, and recovering the variant,
wherein step b) comprises:
i) insertion of a mutation A of at least one amino acid selected from amino acids in position 240, 241, 242, 243, 258, 259, 260, 261, 262, 263, 264, 265, 266, 267, 290, 291, 292, 293, 294, 295, 296, 298, 299, 300, 301, 302, 303, 304 or 305;
ii) insertion of a mutation B selected from the group consisting of 378V, 378T, 434Y and 434S and
   insertion at least one mutation C selected from the group consisting of 226G, 228L, 228R, 230S, 230T, 230L, 241L, 264E, 307P, 315D, 330V, 362R, 378T, 389T, 389K and 434S;
it being understood that:
the mutations are carried out on the Fc fragment of the polypeptide,
the mutations A, B and C do not occur on the same amino acid as mutation B, and the amino acid position numbering of the Fc fragment is that of the EU index or equivalent in Kabat.

Preferably, such a production method comprises, in step b):
i) insertion of a mutation A selected from del294 and 264E;
ii) insertion of a mutation B selected from 378V and 434Y, and insertion of at least one C mutation selected from 315D and 330V,
it being understood that:
the mutations are carried out on the Fc fragment of the polypeptide,
the mutations A, B and C do not take place on the same amino acid, and the amino acid position numbering of the Fc fragment is that of the EU index or equivalent in Kabat.

More preferably, such a production method comprises, in step b), the insertion of a combination of mutations chosen from 259I/315D/434Y and 315D/330V/361D/378V/434Y, it being understood that the number of amino acid positions of the Fc fragment is that of the EU index or equivalent in Kabat.

Preferably, the production method according to the invention is such that step i) is a deletion step of the amino acid in position 294, and step ii) consists in the insertion of a combination of mutations 315D/330V/361D/378V/434Y.

The polynucleotide encoding the variant obtained in step b) may also comprise optimized codons, in particular for its expression in certain cells (step c)). For example, the cells include YB2/0 cells, COS cells, CHO cells, HEK cells, BHK cells, PER.C6 cells, HeLa cells, NIH/3T3 cells, 293 (ATCC #CRL1573), T2 cells, dendritic cells or monocytes. Codon optimization aims to replace natural codons by codons whose transfer RNA (tRNA) carrying the amino acids are most common in the cell type considered. The mobilization of frequently encountered tRNAs has the major advantage of increasing the translation speed of the messenger RNAs (mRNA) and therefore of increasing the final titre (J. M. Carton et al., Protein Expr Purif, 2007). Codon optimization also plays on the prediction of mRNA secondary structures that could slow down reading by the ribosomal complex. Codon optimization also has an impact on the percentage of G/C that is directly related to the half-life of the mRNAs and therefore to their translation potential (Chechetkin, J. of Theoretical Biology 242, 2006 922-934).

Codon optimization may be done by substitution of natural codons using codon frequency (Codon Usage Table) tables for mammals and more specifically for Homo sapiens. There are algorithms available on the internet and made available by the suppliers of synthetic genes (DNA2.0, GeneArt, MWG, Genscript) that make this sequence optimization possible. Preferably, the polynucleotide comprises codons optimized for expression in HEK cells, such as HEK293 cells, CHO cells, or YB2/0 cells. More preferably, the polynucleotide comprises codons optimized for its expression in YB2/0 cells. Alternatively, preferably, the polynucleotide comprises codons optimized for its expression in the cells of transgenic animals, preferably the goat, the rabbit, the ewe or the cow.

### Method for Producing a Variant

In another aspect, the invention also relates to a method for producing a variant of a parent polypeptide comprising an Fc fragment, the variant having an improved half-life relative to the parent polypeptide, comprising the steps of:
i) insertion of a mutation A of at least one amino acid selected from amino acids in position 240, 241, 242, 243, 258, 259, 260, 261, 262, 263, 264, 265, 266, 267, 290, 291, 292, 293, 294, 295, 296, 298, 299, 300, 301, 302, 303, 304 or 305;
ii) insertion of a B mutation selected from the group consisting of 378V, 378T, 434Y and 434S and insertion of at least one C mutation selected from the group consisting of 226G, 228L, 228R, 230S, 230T, 230L, 241L, 264E, 307P, 315D, 330V, 362R, 378V, 378T, 389T, 389K, 434Y and 434S; it being understood that:
   the mutations are made on the Fc fragment of the parent polypeptide,
   the mutations A, B and C do not take place on the same amino acid, and the amino acid position numbering of the Fc fragment is that of the EU index or equivalent in Kabat.

All previously mentioned technical features are applicable here.

### Pharmaceutical Composition

The variant obtained according to the invention may be combined with pharmaceutically acceptable excipients, and optionally extended release matrices, such as biodegradable polymers, to form a therapeutic composition.

The pharmaceutical composition may be administered orally, sublingually, subcutaneously, intramuscularly, intravenously, intraarterially, intrathecally, intraocularly, intracerebrally, transdermally, pulmonally, locally or rectally. The active ingredient, alone or in combination with another active ingredient, may then be administered in unit dosage form, in admixture with conventional pharmaceutical carriers. Unit dosage forms include oral forms such as tablets, capsules, powders, granules and oral solutions or suspensions, sublingual and oral forms of administration, aerosols, subcutaneous implants, transdermal, topical, intraperitoneal, intramuscular, intravenous, subcutaneous, intrathecal, intranasal administration forms and rectal administration forms.

Preferably, the pharmaceutical composition contains a pharmaceutically acceptable carrier for a formulation that may be injected. It may be, in particular, isotonic, sterile, saline solutions (with monosodium or disodium phosphate, sodium chloride, potassium chloride, calcium or magnesium chloride and the like, or mixtures of such salts), or freeze-dried compositions which, when adding sterilized water or physiological saline as appropriate, allow the constitution of injectable solutions.

Dosage forms suitable for injectable use include sterile aqueous solutions or dispersions, oily formulations, including sesame oil, peanut oil, and sterile powders for the extemporaneous preparation of sterile injectable solutions or dispersions. In all cases, the form must be sterile and must be fluid to the extent that it must be injected by syringe. It must be stable under the conditions of manufacture and storage and must be preserved against the contaminating action of microorganisms, such as bacteria and fungi.

The dispersions according to the invention may be prepared in glycerol, liquid polyethylene glycols or mixtures thereof, or in oils. Under normal conditions of storage and use, these preparations contain a preservative to prevent the growth of microorganisms.

The pharmaceutically acceptable carrier may be a solvent or dispersion medium containing, for example, water, ethanol, a polyol (e.g. glycerin, propylene glycol, polyethylene glycol, and the like), suitable mixtures of these, and/or vegetable oils. The proper fluidity may be maintained, for example, by the use of a surfactant, such as lecithin. Prevention of the action of microorganisms may be caused by various antibacterial and antifungal agents, for example, parabens, chlorobutanol, phenol, sorbic acid or thimerosal. In many cases, it will be preferable to include isotonic agents, for example, sugars or sodium chloride. Prolonged absorption of the injectable compositions may be caused by the use in the compositions of agents delaying absorption, for example, aluminum monostearate or gelatin.

Sterile injectable solutions are prepared by incorporating the active ingredients in the required amount in the appropriate solvent with several of the other ingredients listed above, if appropriate, followed by sterilization by filtration. In general, the dispersions are prepared by incorporating the various sterilized active ingredients into a sterile vehicle that contains the basic dispersion medium and the other required ingredients from those enumerated above. In the case of sterile powders for the preparation of sterile injectable solutions, the preferred methods of preparation are vacuum drying and lyophilization. During formulation, the solutions will be administered in a manner compatible with the dosage formulation and in a therapeutically effective amount. The formulations are easily administered in a variety of dosage forms, such as the injectable solutions described above, but drug release capsules and the like may also be used. For parenteral administration in an aqueous solution for example, the solution should be suitably buffered and the liquid diluent rendered isotonic with sufficient saline or glucose. These particular aqueous solutions are particularly suitable for intravenous, intramuscular, subcutaneous and intraperitoneal administration. In this regard, sterile aqueous media that may be used are known to those skilled in the art. For example, a dose may be dissolved in 1 ml of isotonic NaCl solution and then added to 1000 ml of appropriate liquid, or injected at the proposed site of the infusion. Certain dosage variations will necessarily occur depending on the condition of the subject being treated.

The level of a therapeutically effective dose specific for a particular patient will depend on a variety of factors, including the disorder being treated and the severity of the disease, the activity of the specific compound employed, the specific composition used, the age, the body weight, general health, sex and diet of the patient, the time of administration, the route of administration, the rate of excretion of the specific compound used, the duration of treatment, or the drugs used in parallel.

### EXAMPLES

The following examples are given to illustrate various embodiments of the invention.

### Example 1: Production of Variants According to the Invention

The development of the variants of the invention “optimized FcRn” may be done according to the methods described in the prior art, in particular the European patent application EP 0 233 500, which describes the production of such mutants according to the. so-called MutaGen™ Technique.

Typically, this method includes the following steps:

### A/Building a Bank of Fc

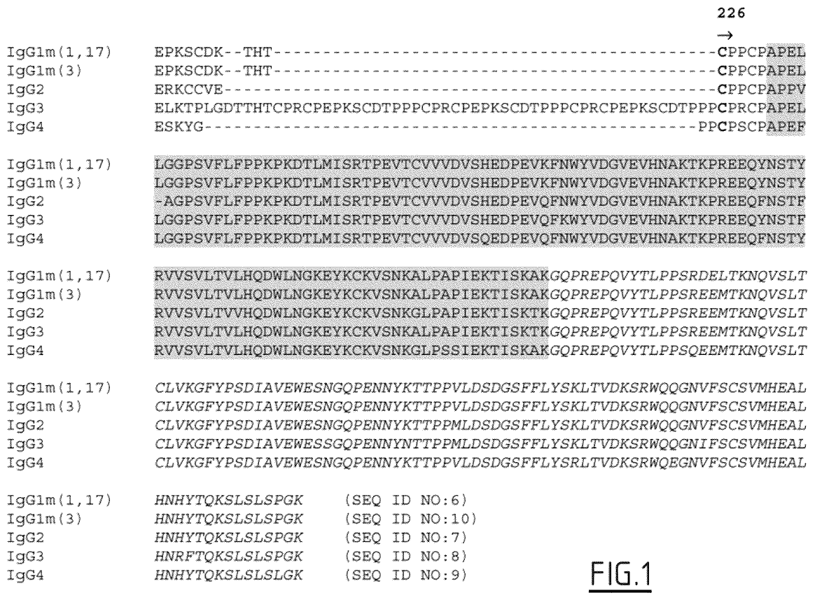

The human Fc gene coding for residues 226 to 447 (according to the EU index of Kabat and represented in FIG. 1) derived from the heavy chain of a human IgG1 is cloned in a suitable vector, such as the phagemid vector pMG58 according to standard protocols well known to those skilled in the art.

### B/Mutagenesis

Several libraries are then generated, according to the procedure described in WO 02/038756, which uses low fidelity human DNA polymerases in order to introduce random mutations homogeneously on the entire target sequence. Specifically, three distinct mutases (pol β, η and ι) were used under different conditions to create complementary mutation profiles.

C/Expression of Fc Banks by Phage-Display and Selection of Variants with Improved Half-Life

The Fc libraries are expressed using the Phage-display technique according to standard protocols, for use in the selection of Fc fragments. Selection may be in accordance with known half-life measurement protocols.

This method allowed the identification of Fc variants of interest with improved FcRn binding, and characterized, such as T5A-74 (N315D/A330V/N361D/A378V/N434Y) and C6A-74 (259I/315D/434Y).

### D/Production of Variants According to the Invention in the Form of Whole Ig

Several combinations of mutations have been selected to serve as a basis for the production of mutants according to the invention.

The following combinations according to the invention have been selected:
T5A-74Del294: E294del/N315D/A330V/N361D/A378V/N434Y
T5A-74H: V264E/N315D/A330V/N361D/A378V/N434Y
but also:
C6A-74Del294: 259I/del294/315D/434Y

### 1—Production of IgG Variants in HEK Cells

The fragment sequence Fc SEQ ID NO: 1 was cloned into a generic eukaryotic expression vector derived from pCEP4 (Invitrogen) and containing the heavy chain of a chimeric anti-CD20 antibody according to standard PCR protocols. The light chain of this antibody was inserted into a similar pCEP4 derived vector. All mutations of interest in the Fc fragment were inserted into the expression vector containing the anti-CD20 heavy chain by overlapping PCR.

For example, the Del294 variant was obtained using two sets of primers adapted to integrate the 294 deletion on the heavy chain contained in the expression vector.

The fragments thus obtained by PCR were combined and the resulting fragment was amplified by PCR using standard protocols. The PCR product was purified on 1% (w/v) agarose gels, digested with the appropriate restriction enzymes and cloned into the anti-CD20 heavy chain expression vector.

HEK 293 cells were cotransfected with the light chain and heavy chain anti-CD20 IgG expression vectors in equimolar amounts according to standard protocols (Invitrogen). The cells were cultured to produce the antibodies transiently. The antibodies produced could be isolated and purified according to standard techniques of the art, with a view to their characterization.

### 2—Production of IgG Variants in YB2/0 Cells

Fc variants were prepared in a full IgG format in the YB2/0 cell line (ATCC, CRL-1662) with anti-CD20 specificity. For this, the IgG heavy and light chain was cloned in a bicistronic vector HKCD20 optimized for production in YB2/0. The production was carried out in stable pools of YB2/0 cells.

The cell culture production and antibody purification steps were carried out according to standard techniques of the art, with a view to their characterization.

The following polypeptides were developed (Anti-CD20 IgG variants):

| Name | Mutations |
|---|---|
| Anti-CD20 C6A-66 | E294del/T307P/N434Y |
| Anti-CD20 Del294 | Del294 |
| Anti-CD20 T5A-74 | N315D/A330V/N361D/A378V/N434Y |
| Anti-CD20 T5A- | E294del/N315D/A330V/N361D/A378V/N434Y |
| 74Del294 | |
| Anti-CD20 T5A-74H | V264E/N315D/A330V/N361D/A378V/N434Y |
| Anti-CD20 WT | / |

The variant T5A-74H differs from the parent variant T5A-74 by the V264E mutation. The mutant T5A-74Del294 differs from the parent variant T5A-74 by the deletion of the amino acid at position 294.

| Name | Mutations |
|---|---|
| Anti-CD20 C6A-74 | 259I/315D/434Y |
| Anti-CD20 Del294 | Del294 |
| Anti-CD20 C6A-74Del294 | 294del/259I/315D/434Y |
| Anti-CD20 WT | / |

The mutant C6A-74Del294 differs from the parent variant C6A-74 by the deletion of the amino acid at position 294.

### Example 2: Analysis of the Half-Life of IqGs According to the Invention

Pharmacokinetic experiments were thus performed in hFcRn mice that are homozygous KO for a murine and heterozygous FcRn allele for a human FcRn transgene (mFcRn^{-/-} hFcRnTg).

For these pharmacokinetic studies, each animal received a single intravenous injection of 5 mg/kg IgG at the retro-orbital sinus, in a protocol similar to that previously described (Petkova SB, et al., Enhanced half-life of genetically engineered human IgG1 antibodies in a humanized FcRn mouse model: potential application in humorally mediated autoimmune disease (Int Immunol 2006).

Generally the half-life is calculated from the plasma concentrations measured during the elimination phase.

The half-life time may thus be obtained:
by solving the equation:

$$T 1 / 2 = ( Ln 2 \times Vd ) / CL , where \\ Vd = volume of distribution = initial dose / plasma concentration$$

$$CL = Clearance = Dose / AUC ( area under the curve )$$

by graphical analysis by determining on the ordinate axis (concentration in μg/ml) the interval of time elapsed between the concentration C1 and the concentration C2. It is imperative to draw this curve on a semi-logarithmic scale in order to ensure the alignment of the experimental points in this last so-called phase of elimination. The duration of exploration of this slope must be long enough to allow an accurate estimation of the half-life.

Once the slope of the measured elimination phase (kₑ or elimination constant), the half-life may be calculated as follows:

T1/2=Ln2/kₑ=0.693/kₑ

In addition, the MRT or the average residence time may be estimated. It reflects the duration of presence of the Fc polypeptide in the body.

The MRT may be obtained as follows:
MRT=AUC/AUMC, where
AUC=time 0 of the plasma concentration versus time curve
AUMC=first order moment of the plasma concentration versus time curve.

Blood samples were taken from the retro-orbital sinus at multiple time points and IgGs titrated by ELISA.

### Results:

### Variants T5A-74:

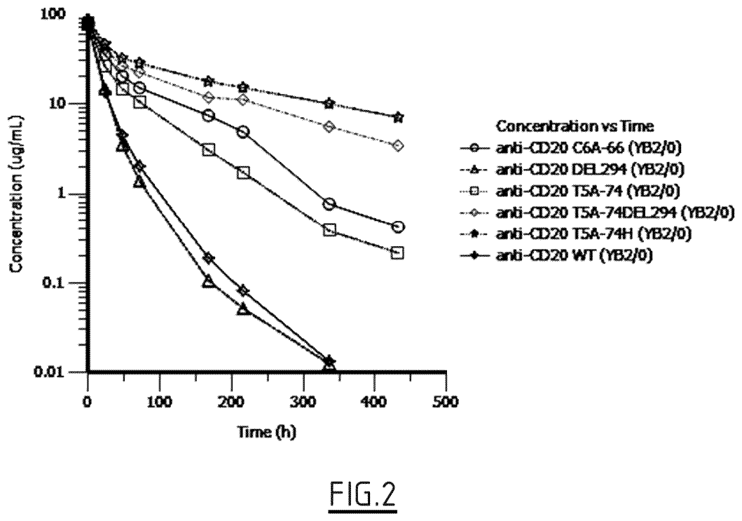

In this assay, both IgG T5A-74 and Del294 showed an increase in half-life (FIG. 2). Advantageously, the combination of mutations T5A-74 and Del 294 on the one hand, and T5A-74 and V264E, allowed one to observe a synergistic effect on the increase of the half-life of IgG T5A-74Del294 and T5A-74H.

The results are summarized in the table below:

| | T1/2 | Cmax | AUCinf | Vd | Cl | MRT |
|---|---|---|---|---|---|---|
| mAb_ID | (h) | (μg/mL) | (h*μg/mL) | (mL/kg) | (mL/h/kg) | (h) |
| anti-CD20 WT (YB2/0) | 34.2 | 72.2 | 1478 | 167 | 3.38 | 17.7 |
| anti-CD20 DEL294 (YB2/0) | 54.4 | 75.3 | 1471 | 267 | 3.40 | 15.0 |
| anti-CD20 C6A66 (YB2/0) | 74.2 | 79.0 | 4261 | 126 | 1.17 | 83.1 |
| anti-CD20 T5A74 (YB2/0) | 64.1 | 76.9 | 3118 | 148 | 1.60 | 64.7 |
| anti-CD20 T5A74H (YB2/0) | 178 | 85.7 | 10234 | 126 | 0.489 | 228 |
| anti-CD20 T5A74DEL294 (YB2/0) | 154 | 81.9 | 7755 | 143 | 0.645 | 191 |

The parameters analyzed are defined below:
T1/2: half-life
Cmax: maximum concentration obtained at a given time, corresponding to time maximum plasma concentration (Tmax)
AUCinf: Area under the time/plasma concentration curve from T0 to infinity
Vd: Distribution volume
Cl: Clearance
MRT: average residence time

### Variants C6A-74:

IgG C6A-74Del294 also shows an increase in half-life. Advantageously, the combination of the C6A-74 and Del 294 mutations made it possible to observe an increase in the half-life.

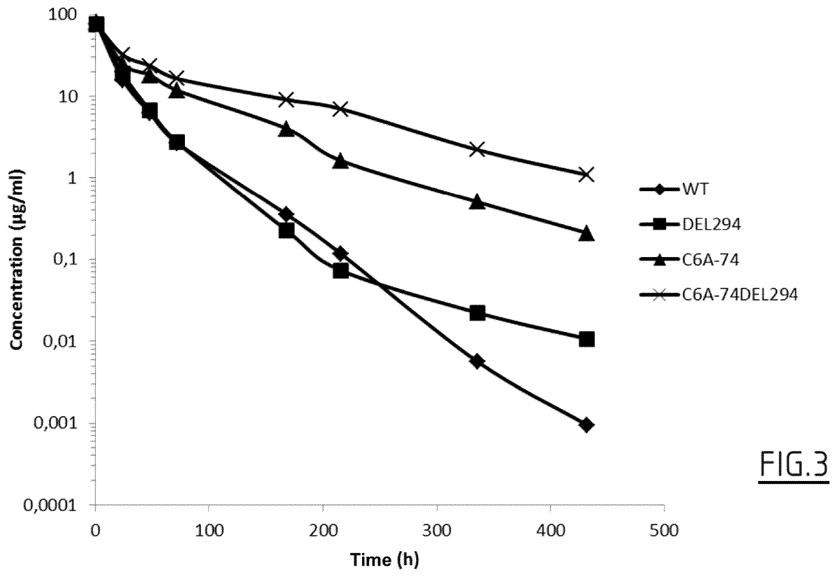

The results are shown in FIG. 3, and are summarized in the table below:

| | T1/2 | Cmax | AUCinf | Vd | CL | MRT |
|---|---|---|---|---|---|---|
| mAb_ID | (h) | (μg/mL) | (h*ug/mL) | (mL/kg) | (mL/h/kg) | (h) |
| anti-CD20 WT (YB2/0) | 34.6 | 76.8 | 1688 | 62.4 | 3.05 | 20.4 |
| anti-CD20 DEL294 (YB2/0) | 74.8 | 76.5 | 1785 | 58.0 | 2.82 | 20.6 |
| anti-CD20 C6A-74 (YB2/0) | 82.8 | 79.6 | 3432 | 119.8 | 1.52 | 81.1 |
| anti-CD20 C6A-74DEL294 (YB2/0) | 116 | 74.1 | 5710 | 128 | 0.897 | 145 |

The parameters analyzed are defined below:
T1/2: half-life
Cmax: maximum concentration obtained at a given time, corresponding to time maximum plasma concentration (Tmax)
AUCinf: Area under the time/plasma concentration curve from T0 to infinity
Vd: Distribution volume
Cl: Clearance
MRT: average residence time

### Example 3: Analysis of the Impact of IqG Sialylation According to the Invention

The same pharmacokinetic parameters as those studied in Example 2 were studied for the desialylated Del294, T5A_74, T5A_74Del294 and T5A_74Del294 IgGs, according to the same method as that described in Example 2.

The desialylated T5A_74Del294 IgGs are prepared as follows: 4 mg of samples to be desialylated were incubated with 160 μL of Sialidase A (such as GK80040 from Prozyme) for 24 h at 37° C. The samples were then purified on protein A, dialyzed in PBS and concentrated on Vivaspin 30 kDa and sterile filtered.

### Variants T5A-74:

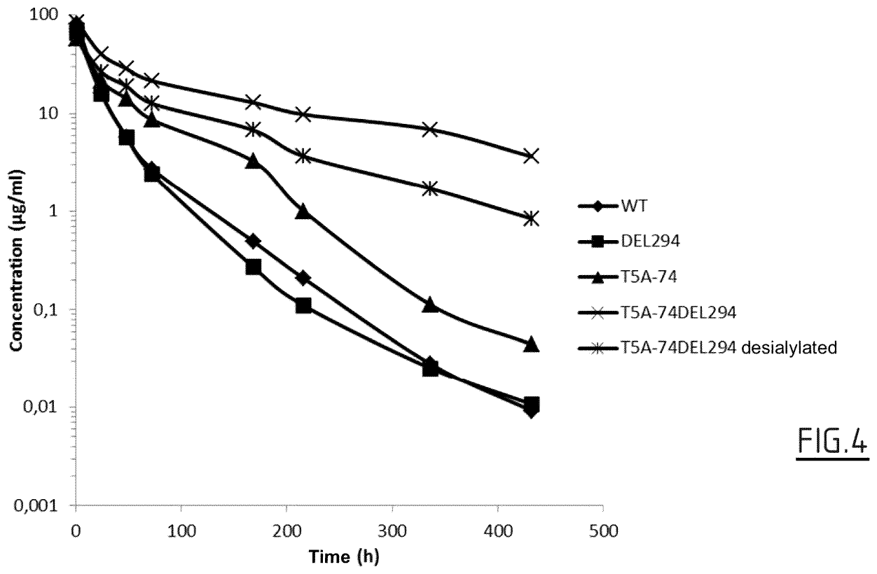

The results obtained are illustrated in FIG. 4, and are summarized in the table below:

| | T1/2 | Cmax | AUCinf | Vd | CL | MRTinf |
|---|---|---|---|---|---|---|
| Molecule | (h) | (μg/ml) | (h*μg/ml) | (ml/kg) | (ml/h/kg) | (h) |
| WT | 45.8 | 79.8 | 1739 | 67.1 | 2.90 | 23.1 |
| DEL294 | 68.1 | 66.2 | 1516 | 74.8 | 3.35 | 22.3 |
| T5A-74 | 53.4 | 57.7 | 2430 | 124.0 | 2.25 | 56.4 |
| T5A-74DEL294 | 153.0 | 84.5 | 7681 | 122.0 | 0.67 | 195.0 |
| T5A-74DEL294 | 70.5 | 56.0 | 3689 | 138.0 | 1.43 | 103.0 |
| desialylated | | | | | | |

These results show that a sialylation-increasing mutation such as Del294 (see Example 6) gives a good half-life.

The only mutation Del294 increases the half-life, but not the MRT compared to the WT. On the other hand, the half-life and the MRT are both increased with the variant T5A_74Del294 (compared to Del294, but also to WT). In particular, the half-life of T5A_74Del294 is very strongly increased, compared with that of the Del294 mutant or the T5A-74 mutant.

Finally, the half-life and the MRT are decreased with the variant T5A_74Del294 desialylated with respect to the variant T5A_74Del294, which confirms the impact of the sialylation.

### Example 4: Binding on hFcRn Cells

FcRn receptor binding was investigated by a competitive assay using A488-labeled Rituxan and Jurkat cells stably expressing human FcRn (hFcRn) at the surface (Jurkat-FcRn).

The Jurkat-FcRn cells were incubated for 20 minutes at 4° C. with variable concentrations (500; 250; 125; 62; 31; 15; 8; 4; 2; 0 μg/ml) of antibodies Del294, T5A_74, T5A_74Del294., Desialylated T5A_74Del294, C6A_74, C6A_74Del294 and desialylated C6A_74Del294, diluted in PBS at pH6, simultaneously with Rituxan-A488 used at a fixed concentration.

After washing, the binding of Rituxan-A488 to FcRn expressed by Jurkat-FcRn cells was evaluated by flow cytometry. The mean fluorescence values (MFI) observed are expressed as a percentage, 100% being the value obtained with Rituxan-A488 alone and 0% the value obtained in the absence of Rituxan-A488.

### Variants T5A-74:

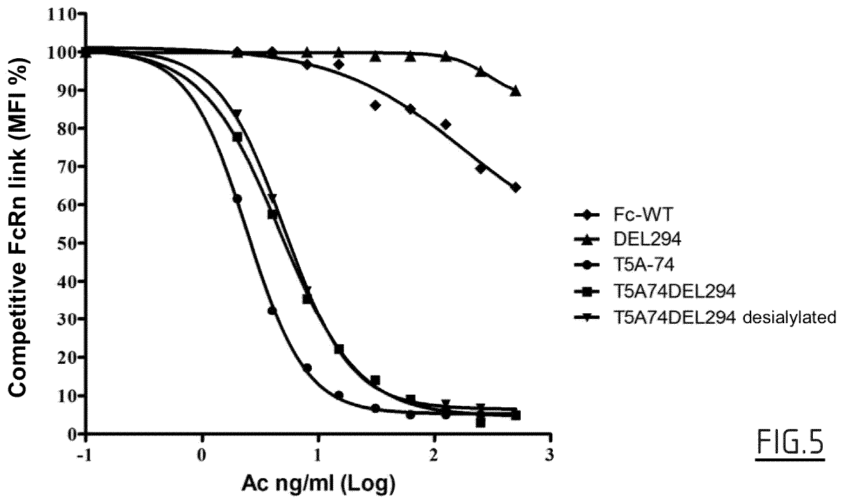

The results obtained are illustrated in FIG. 5, and are summarized in the table below:

| Molecule | EC50 (nM) |
|---|---|
| WT | >500 |
| DEL294 | >500 |
| T5A-74 | 2.5 |
| T5A-74DEL294 | 4.8 |
| T5A-74DEL294 desialylated | 5.2 |

### Variants C6A-74:

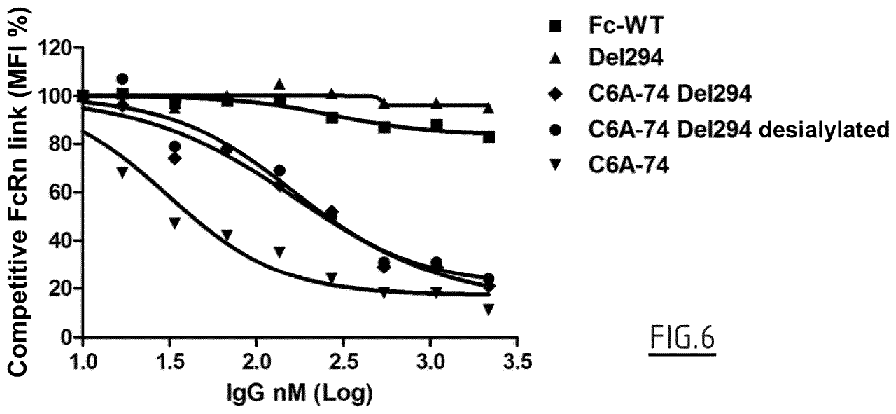

The results obtained are illustrated in FIG. 6, and are summarized in the table below:

| Molecule | EC50 (nM) |
|---|---|
| WT | 280.3 |
| DEL294 | >500 |
| C6A-74 | 31.15 |
| C6A-74DEL294 | 172.4 |
| C6A-74DEL294 desialylated | 162.2 |

These results show a small decrease in hFcRn binding induced by the addition of the Del294 mutation. These variations remain very small, compared to the increases obtained by the mutations specific to T5A_74 (i.e. N315D, A330V, N361D, A378V and N434Y) and to C6A_74 (i.e. 259I, 315D and 434Y), and are inverse to what is observed in vivo.

In all cases, these tests show an equivalent or weakly diminished binding to hFcRn, and no significant increase in binding to hFcRn.

### Example 5: Links on CD20 Cells

The CD20-expressing Raji cells and the desialylated Tc174, T5A_74, T5A_74Del294,T5A_74Del294, C6A_74, C6A_74Del294 and C6A_74Del294 antibodies were diluted in PBS with 1% FCS.

1×10⁵ cells were incubated with 100 μl of antibodies (anti-CD20 variants Del294, T5A_74, T5A_74Del294, desialylated T5A_74Del294, C6A_74, C6A_74Del294, desialylated C6A_74Del294, or negative control) at different final concentrations (0; 0.1; 0.5; 2, 10 μg/ml) at 4° C. on ice for 20 minutes.

After washing with the diluent, the antibodies were visualized with a goat F(ab′)2 anti-Fc fragment of human IgG, coupled with phycoerythrin (for example, Ref: Jackson 109-116-098 lot 122690, 100 μl of a dilution of 1:100 in diluent) at 4° C. on ice for 20 minutes. The cells were washed and the mean fluorescence intensity was evaluated with an Flow cytometer (FC500, Beckman Coulter). The arbitrary values of Kd were calculated using PRISM software.

### Variants T5A-74:

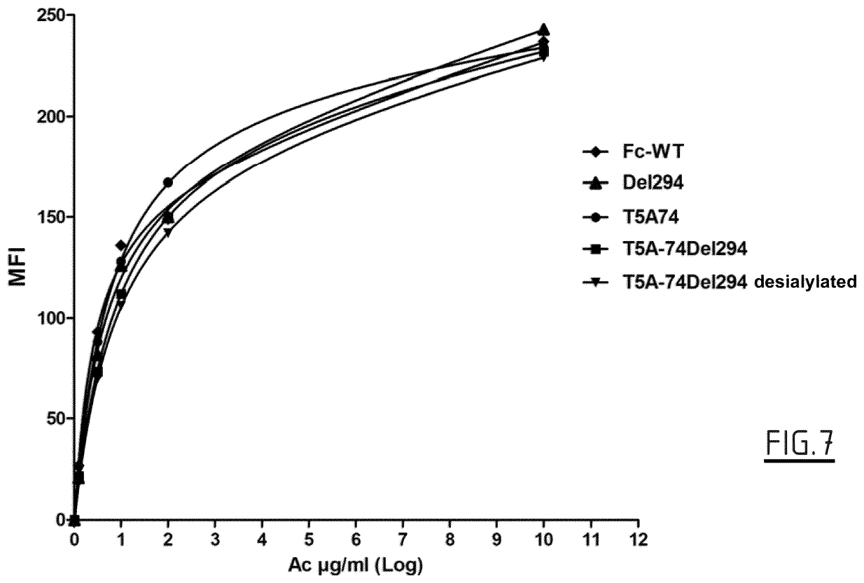

The results obtained are illustrated in FIG. 7, and are summarized in the table below:

| Molecule | Bmax MFI | Kd (μg/ml) |
|---|---|---|
| WT | 176 | 0.4 |
| DEL294 | 187 | 0.6 |
| T5A-74 | 225 | 0.8 |
| T5A-74DEL294 | 207 | 0.9 |
| T5A-74DEL294 desialylated | 193 | 0.9 |

### Variants C6A-74:

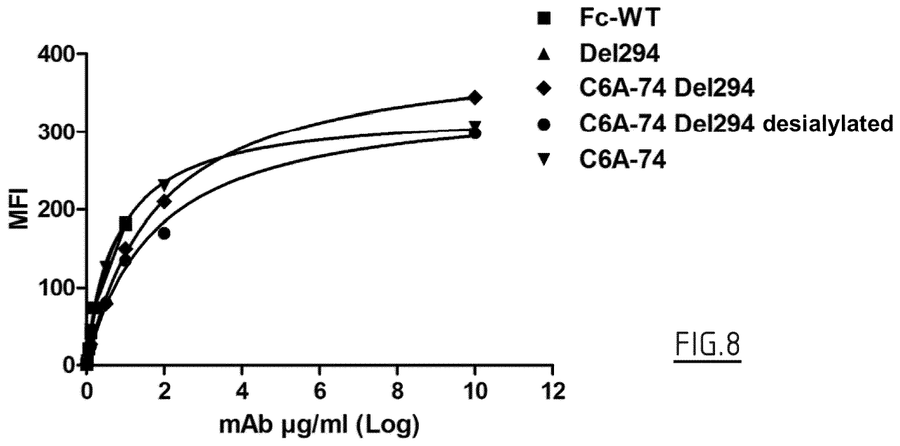

The results obtained are illustrated in FIG. 8, and are summarized in the table below:

| Molecule | Bmax MFI | Kd (μg/ml) |
|---|---|---|
| WT | 286.3 | 0.8 |
| DEL294 | 263.1 | 0.8 |
| C6A-74 | 327.5 | 0.9 |
| C6A-74DEL294 | 407.3 | 1.3 |
| C6A-74DEL294 desialylated | 346.3 | 1.7 |

The results show a good CD20 binding, regardless of the variants. Mutations, alone or in combination, do not affect the binding of the mutated antibody to its antigen. There is therefore no impact on the in vivo efficacy depending on the antigen binding.

### Example 6: Study of the Sialylation by HPCE-Lif of the T5A 74Del294 and T5A 74H Variants

The following polypeptides produced in YB2/0 (obtained in Example 1.2) were analyzed:

| Name | Mutations |
|---|---|
| Anti-CD20 WT | |
| Anti-CD20 Del294 | Del294 |
| Anti-CD20 T5A_74- | E294del/N315D/A330V/N361D/A378V/N434Y |
| Del294 | |
| Anti-CD20 T5A_74H | V264E/N315D/A330V/N361D/A378V/N434Y |

### Operating Mode: Preparation of the Sample

### 1 Desalting and N-deglycosylation

Firstly, the sample to be analyzed was desalinated according to standard protocols in order to eliminate all the free reducing glucides potentially present as well as substances that could interfere during the subsequent steps (salts and excipients). After desalting, the sample was dried and the glycols were released by the enzymatic action of N-Glycannase under denaturing and reducing conditions, in order to maximize the yield of N-deglycosylation. For N-deglycosylation of Ig, the dry sample was taken up in 45 μl of the PNGase F digestion solution diluted 1/5. 1.5 μl of a solution of β-mercaptoethanol 10% (v/v) in ultrapure water was added before stirring and incubation for 15 minutes at room temperature. Then, 1 μL of the PNGase F solution (2.5 mU/μL) was added before stirring and incubation in a water bath at 37° C. for 12 to 18 hours. The glycans were then separated from the deglycosylated proteins by precipitation with cold EtOH.

The glycan extract obtained was then divided into 4 fractions before being treated with exoglycosidases.

Each dried alcohol N subfraction, containing the equivalent of 100 μg of glycoprotein, was respectively digested (1) by α-sialidase, β-galactosidase and N-acetyl-β-hexosaminidase, in order to determine the fucosylation rate; (2) α-sialidase, β-galactosidase and α-fucosidase, for calculating the level of GlcNAc intercalary; and (3) α-sialidase and α-fucosidase, to determine the galactosylation index.

These deglycosylations were carried out at 37° C. for 12 to 18 hours.

The isolation of the exoglycosidic degradation products was carried out by cold alcohol extraction by adding 60 μl (3 volumes) of absolute ethanol equilibrated at −20° C., before stirring and then incubation at −20° C. for 15 minutes. Centrifugation at 10,000 rpm was performed for 10 minutes at +4° C., and the supernatant was immediately transferred to a 0.5 mL microtube before being dried under vacuum.

The oligosaccharides obtained were then labeled with an Fluorochrome, the APTS, then separated and quantified in HPCE-LIF.

### 2 Use of the Results

The identification of the N-glycanse peaks is carried out using a standard glycoprotein standard whose N-glycosylation is perfectly known, by comparing the migration times of its N-glycans with those of the species observed on the electrophoretic profiles of the samples to be analyzed. In addition, the migration times of the standard oligosaccharides are converted into units of glucose (GUs) after analysis of a heterogeneous mixture of a glucose homopolymer (Glc ladder). These values of GUs will then be compared with those of some standard oligosaccharides of known GUs, and will make it possible to increase the confidence index of the identifications.

The identification of the N-glycans peaks obtained on the different electrophoretic profiles makes it possible to identify a complete glycan profile, and in particular to evaluate the percentage of sialylated forms.

The results are as follows:

| Anti-CD20 WT: | |
|---|---|
| Structure (%) | HPCE-LIF |
| Galactose level = | 40.30 |
| Sialic acid ratio = | 1.81 |
| Mono-sialylated structures | 0.68 |
| Bi-sialylated structures | 0.00 |
| Unidentified sialylated structures | 1.13 |
| GlcNac bisectors = | 2.62 |
| Fucose rate * | 9.24 |
| Fucose rate | 10.29 |
| A2 | 0.00 |
| A2F | 0.00 |
| M3N2 | 0.00 |
| M3N2F | 0.00 |
| A1 | 0.68 |
| A1F | 0.00 |
| G2FB | 0.00 |
| G2F | 0.86 |
| G2B | 0.00 |
| G2 | 4.74 |
| G1FB | 0.00 |
| G1F | 4.15 |
| G1(1.3)FB | 0.00 |
| G1(1.6)FB | 0.00 |
| G1(1.3)F | 0.00 |
| G1(1.6)F | 4.15 |
| G1B | 0.93 |
| G1 | 22.66 |
| G1(1.3)B | 0.00 |
| G1(1.6)B | 0.93 |
| G1(1.3) | 0.39 |
| G1(1.6) | 22.27 |
| G0FB | 0.39 |
| G0F | 4.89 |
| G0B | 1.30 |
| G0 | 50.78 |
| MAN-5 | 0.00 |
| Identified (%) | 99.15 |

* obtained from the run Dsial + Dgal + DHexNAc

The majority of the structures are short afucosylated forms (G0+G1=74.7%). The sialylation rate observed is very low (1.81% of sialic acids).

### Anti-CD20 Del294:

The electrophoregrams obtained show biantennian glycan structures.

These structures are mainly sialylated.

87.98% of the structures seem sialylated.

| | |
|---|---|
| A2 | 11.9 |
| A2F | 19.8 |
| A1 | 5.5 |
| A1F | 1.6 |
| G0 | 0.84 |
| G0B | 0.96 |
| G1(1.6) + G0F | 0.53 |
| G1(1.3) + G0BF | 0.19 |
| G1 (1.6) B | 2.44 |
| G1 (1.6) F | 0.14 |
| G2 + G1(1.3)F | 2.09 |
| G2B | 0.51 |
| G2F | 0.16 |
| G2FB | 3.84 |
| Unidentified sialylated structures | 49.18 |
| % age of sialylated structures | 87.98 |
| % age of fucosylation | 48.24 |

### Anti-CD20 T5A-74Del294:

The electrophoregrams of the native and desialylated structures of the tested sample show that 96.5% of the structures are sialylated.

### Anti-CD20 T5A-74H:

The electrophoregrams of the native and desialylated structures of the test sample show that 100% of the structures have at least one sialic acid residue.

In conclusion, the WT YB2/0 molecule exhibits a classical glycosylation profile in YB2/0, with a low sialylation profile.

The T5A-74H and T5A_74Del294 molecules have very similar profiles: they essentially comprise sialylated structures.

The strong sialylation observed for the del294 deletion alone or for the V264E mutation alone (not shown) is not affected when these single mutations are combined with those of a mutant such as T5A-74.

1. Variant of a parent polypeptide comprising an Fc fragment, the variant having an improved half-life relative to the parent polypeptide, and comprising:
At least one mutation of the Fc fragment increasing the sialylation of the Fc; and
at least one combination of mutations selected from the group consisting of 307A/315D/330V/382V/389T/434Y, 256N/378V/383N/434Y, 315D/330V/361D/378V/434Y, 259I/315D/434Y, 230S/315D/428L/434Y, 241L/264E/307P/378V/433R, 250A/389K/434Y, 305A/315D/330V/395A/434Y, 264E/386R/396L/434S/439R, 315D/330V/362R/434Y, 294del/307P/434Y, 305A/315D/330V/389K/434Y, 315D/327V/330V/397M/434Y, 230T/241L/264E/265G/378V/421T, 264E/396L/415N/434S, 227L/264E/378V/434S, 264E/378T/396L, 230T/315D/362R/426T/434Y, 226G/315D/330V/434Y, 230L/241L/243L/264E/307P/378V, 250A/315D/325S/330V/434Y, 290E/315D/342R/382V/434Y, 241L/315D/330V/392R/434Y, 241L/264E/307P/378V/434S, 230T/264E/403T/434S, 264E/378V/416K, 230T/315D/362E/434Y, 226G/315D/434Y, 226G/315D/362R/434Y, 226G/264E/347R/370R/378V/434S, 3081/315D/330V/382V/434Y, 230T/264E/378V/434S, 231T/241L/264E/378T/397M/434S, 230L/264E/378V/434S, 230T/315D/330V/386K/434Y, 226G/315D/330V/389T/434Y, 267R/307P/378V/421T/434Y, 230S/315D/387T/434Y, 230S/264E/352S/378V/434S and 230T/303A/322R/389T/404L/434S, it being understood that the mutation of the Fc fragment increasing the sialylation of the Fc cannot take place on the same amino acid as one of the amino acids of said combination of mutations, and
that the amino acid position numbering of the Fc fragment is that of the EU index or equivalent in Kabat.

2. Variant according to claim 1, comprising;
i) a mutation A of at least one amino acid selected from amino acids in position 240, 241, 242, 243, 258, 259, 260, 261, 262, 263, 264, 265, 266, 267, 290, 291, 292, 293, 294, 295, 296, 298, 299, 300, 301, 302, 303, 304 or 305; and
ii) at least one combination of mutations selected from 307A/315D/330V/382V/389T/434Y, 256N/378V/383N/434Y, 259I/315D/434Y, 230S/315D/428L/434Y, 294del/307P/434Y and 315D/330V/361D/378V/434Y, it being understood that mutation A can not take place on the same amino acid as one of the amino acids of mutation ii), and
that the amino acid position numbering of the Fc fragment is that of the EU index or equivalent in Kabat.

3. Variant according to claim 1, wherein the mutation A is del294 or 264E, it being understood that the amino acid position numbering of the Fc fragment is that of the EU index or equivalent in Kabat.

4. Variant according to claim 1, wherein the parent polypeptide consists of an Fc fragment.

5. Variant according to claim 1, wherein the parent polypeptide is an immunoglobulin or an antibody.

6. Variant according to claim 1, wherein the Fc fragment of the parent polypeptide is an Fc fragment of an IgG.

7. Variant according to claim 1, wherein the half-life of the variant is increased by a factor at least equal to 2 relative to the parent polypeptide.


SEQUENCE LISTING (10 sequences)

SEQ ID NO: 1 - protein, 222 aa, synthetic construct
  REGION 1..222: note = Fc of IgG1 G1m1,17
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 221 aa, synthetic construct
  REGION 1..221: note = Fc of IgG2
CPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 3 - protein, 222 aa, synthetic construct
  REGION 1..222: note = Fc of IgG3
CPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 4 - protein, 222 aa, synthetic construct
  REGION 1..222: note = Fc of IgG4
CPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 222 aa, synthetic construct
  REGION 1..222: note = Fc of IgG1 G1m3
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 232 aa, synthetic construct
  REGION 1..232: note = Fc of IgG1 G1m1,17
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 228 aa, synthetic construct
  REGION 1..228: note = Fc of IgG2
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 8 - protein, 279 aa, synthetic construct
  REGION 1..279: note = Fc of IgG3
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 9 - protein, 229 aa, synthetic construct
  REGION 1..229: note = Fc of IgG4
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 232 aa, synthetic construct
  REGION 1..232: note = Fc of IgG1 G1m3
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK